(19) United States
(12) Patent Application Publication
(10) Pub. No.: US 2026/0363756 A1
(43) Pub. Date: Nov. 19, 2026

(54) END EFFECTOR ATTACHMENT ACCESSORY AND RELATED METHODS

(71) Applicant: Cilag GmbH International, Zug (CH)

(72) Inventors: Michael J. Vendely, Lebanon, OH (US); Nohemi C. Wells, Cincinnati, OH (US); Christopher J. Waid, Norwood, OH (US); Madeleine C. Jayme, Cincinnati, OH (US); Jeffrey L. Clark, Maineville, OH (US)

(21) Appl. No.: 19/212,064

(22) Filed: May 19, 2025

(51) Int. Cl. A61B 18/14; A61B 18/00

(52) U.S. Cl. CPC A61B 18/1445; A61B 2018/00077; A61B 2018/00083; A61B 2018/00172; A61B 2018/00184; A61B 2018/00601; A61B 2018/1452

(57) ABSTRACT

A surgical instrument and related methods includes an accessory tool for attachment to an instrument end effector. The accessory tool includes a shroud, a coupling adapter proximally extending from the shroud, and a tool end effector distally extending from the shroud. The coupling adapter includes an upper grip portion configured to removably secure with the instrument end effector and an upper grip surface configured to contact an upper electrode of the instrument end effector. The tool end effector includes an upper tool electrode in electrical communication with the upper grip surface.

### BACKGROUND

A variety of medical instruments may be used in procedures conducted by a medical professional operator, as well as applications in robotically assisted surgeries. In the case of robotically assisted surgery, the clinician may operate a master controller to remotely control the motion of such medical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletol gloves, master manipulators, or the like), which are coupled by a servo mechanism to the medical instrument. In some scenarios, a servo motor moves a manipulator supporting the medical instrument based on the clinician's manipulation of the hand input devices. During the medical procedure, the clinician may employ, via a robotic system, a variety of medical instruments including an ultrasonic blade, a surgical stapler, a tissue grasper, a needle driver, an electrosurgical cautery probes, etc. Each of these structures performs functions for the clinician, for example, cutting tissue, coagulating tissue, holding or driving a needle, grasping a blood vessel, dissecting tissue, or cauterizing tissue.

Examples of robotic systems are described in U.S. Pat. No. 9,763,741, entitled “System for Robotic-Assisted Endolumenal Surgery and Related Methods,” issued Sep. 19, 2017, the disclosure of which is incorporated by reference herein, in its entirety; U.S. Pat. No. 10,464,209, entitled “Robotic System with Indication of Boundary for Robotic Arm,” issued Nov. 5, 2019, the disclosure of which is incorporated by reference herein, in its entirety; U.S. Pat. No. 10,667,875, entitled “Systems and Techniques for Providing Multiple Perspectives During Medical Procedures,” issued Jun. 2, 2020, the disclosure of which is incorporated by reference herein, in its entirety; U.S. Pat. No. 10,765,303, entitled “System and Method for Driving Medical Instrument,” issued Sep. 8, 2020, the disclosure of which is incorporated by reference herein, in its entirety; U.S. Pat. No. 10,827,913, entitled “Systems and Methods for Displaying Estimated Location of Instrument,” issued Nov. 10, 2020, the disclosure of which is incorporated by reference herein, in its entirety; U.S. Pat. No. 10,881,280, entitled “Manually and Robotically Controllable Medical Instruments,” issued Jan. 5, 2021, the disclosure of which is incorporated by reference herein, in its entirety; U.S. Pat. No. 10,898,277, entitled “Systems and Methods for Registration of Location Sensors,” issued Jan. 26, 2012, the disclosure of which is incorporated by reference herein, in its entirety; and U.S. Pat. No. 11,058,493, entitled “Robotic System Configured for Navigation Path Tracing,” issued Jul. 13, 2021, the disclosure of which is incorporated by reference herein, in its entirety.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 depicts a perspective view of an example of a table-based robotic system that includes a control console and a plurality of robotic arms;

FIG. 2 depicts a perspective view of an example of a robotic arm, an example of a tool drive, and a first example of a surgical instrument, each configured for use with the table-based robotic system of FIG. 1;

FIG. 3A depicts an enlarged schematic perspective view of the tool driver and the surgical instrument of FIG. 2;

FIG. 3B depicts a schematic perspective view of the tool driver similar to FIG. 3A, but with the surgical instrument removed to expose rotary drives;

FIG. 4 depicts a perspective view of a second example of a surgical instrument configured to use with the table-based robotic system of FIG. 1 as well as the robotic arm and the tool drive of FIG. 2;

FIG. 5 depicts an enlarged perspective view of a distal end portion of the surgical instrument of FIG. 4 with an end effector in an open configuration and an articulation section in a straight configuration;

FIG. 6 depicts an enlarged perspective view of the distal end portion of the surgical instrument of FIG. 4 with the end effector in the open configuration and rolled about a longitudinal axis;

FIG. 7 depicts an enlarged perspective view of the distal end portion of the surgical instrument of FIG. 4 with the end effector in the open configuration and the articulation section articulated to adjust a pitch of the end effector through a pitch plane;

FIG. 8 depicts an enlarged perspective view of the distal end portion of the surgical instrument of FIG. 4 with the end effector in the open configuration and the articulation section articulated to adjust a yaw of the end effector through a yaw plane;

FIG. 9 depicts an enlarged perspective view of the distal end portion of the surgical instrument of FIG. 4 with the end effector in a closed configuration and the yaw of the end effector adjusted through the yaw plane;

FIG. 10 depicts a perspective view of an example of a first tool accessory;

FIG. 11 depicts a top view of the first tool accessory;

FIG. 12 depicts an enlarged sectional view of the distal end portion of the surgical instrument of FIG. 4 with the end effector grasping the first tool accessory of FIG. 10;

FIG. 13 depicts an enlarged sectional view of the distal end portion of the surgical instrument of FIG. 4 with an example of a second tool accessory affixed to the end effector;

FIG. 14 depicts an enlarged top view of the distal end portion of the surgical instrument of FIG. 4 with an example of a third tool accessory affixed to the end effector;

FIG. 15 depicts an enlarged side view of the distal end portion of the surgical instrument of FIG. 4 with the third tool accessory of FIG. 14 affixed to the end effector;

FIG. 16 depicts a top view of an example of a fourth tool accessory;

FIG. 17 depicts a top view of an example of a fifth tool accessory;

FIG. 18 depicts a top view of an example of a sixth tool accessory;

FIG. 19 depicts a top view of an example of a seventh tool accessory;

FIG. 20 depicts a top view of an example of an eighth tool accessory;

FIG. 21 depicts a top view of an example of a ninth tool accessory;

FIG. 22 depicts a top view of an example of a tenth tool accessory;

FIG. 23 depicts a top view of an example of an eleventh tool accessory;

FIG. 24A depicts an enlarged sectional view of the distal end portion of the surgical instrument of FIG. 4 with the first tool accessory of FIG. 10 positioned between the end effector in the open configuration;

FIG. 24B depicts an enlarged sectional view of the distal end portion of the surgical instrument of FIG. 4 with the first tool accessory of FIG. 10 positioned between the end effector transitioning from the open configuration to the closed configuration; and

FIG. 24C depicts an enlarged sectional view of the distal end portion of the surgical instrument of FIG. 4 with the first tool accessory of FIG. 10 positioned between the end effector in the closed configuration.

### DETAILED DESCRIPTION

### I. Overview of Example of Robotic Surgical System

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Example of Robotic System Table

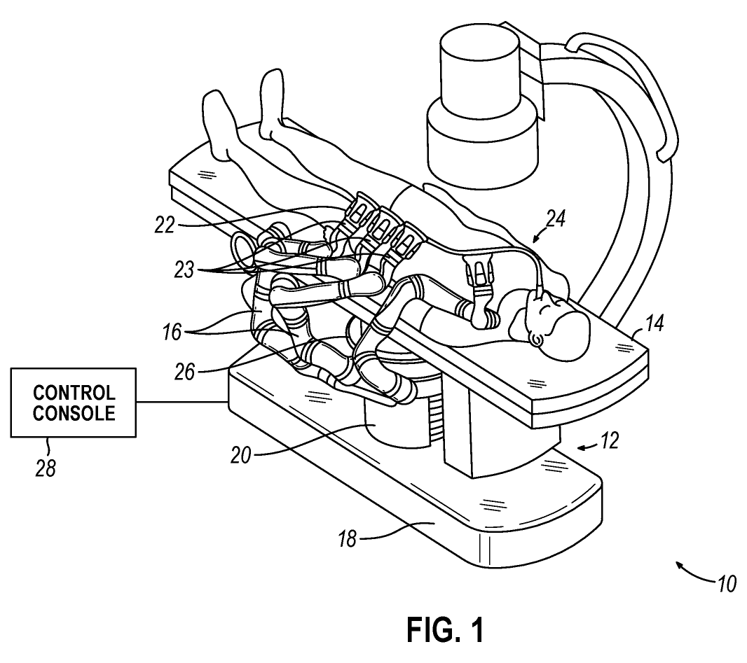

FIG. 1 illustrates an example of a robotic surgical system (10). Robotic surgical system (10) includes a support structure (12) for supporting a platform (14) (shown as a “table” or “bed”) over the floor and one or more robotic arms (16). Support structure (12) includes a base (18) and a column (20). Column (20) structurally supports platform (14), and provides a path for vertical translation of the carriages. In some versions, a table base may stow and store robotic arms (16) when not in use. Column (20) of the present example also includes a ring-shaped carriage (26), from which robotic arms (16) are based. A control console (28) is coupled with robotic surgical system (10). While four robotic arms are shown, more or fewer robotic arms are envisioned.

Robotic arms (16) are shown as part of a table-mounted system, but in other configurations, robotic arms (16) may be mounted in a cart, ceiling or sidewall, or other suitable support surface. Robotic arms (16) are shown as extending from column (20) via carriage (26). However, robotic arms (16) may be coupled with robotic surgical system (10) using a variety of suitable structures. While robotic arms (16) are all shown as being positioned on one side of the patient in FIG. 1, other configurations may position robotic arms (16) on both sides of the patient, between the legs of the patient, and/or in any other suitable locations. Tool drivers (22) are positioned at distal ends of robotic arms (16) in the present example. Tool drivers (22) are operable to manipulate one or more surgical instruments (24), as will be described in greater detail below.

### B. Example of a Robotic Arm, Tool Drive, and Tool

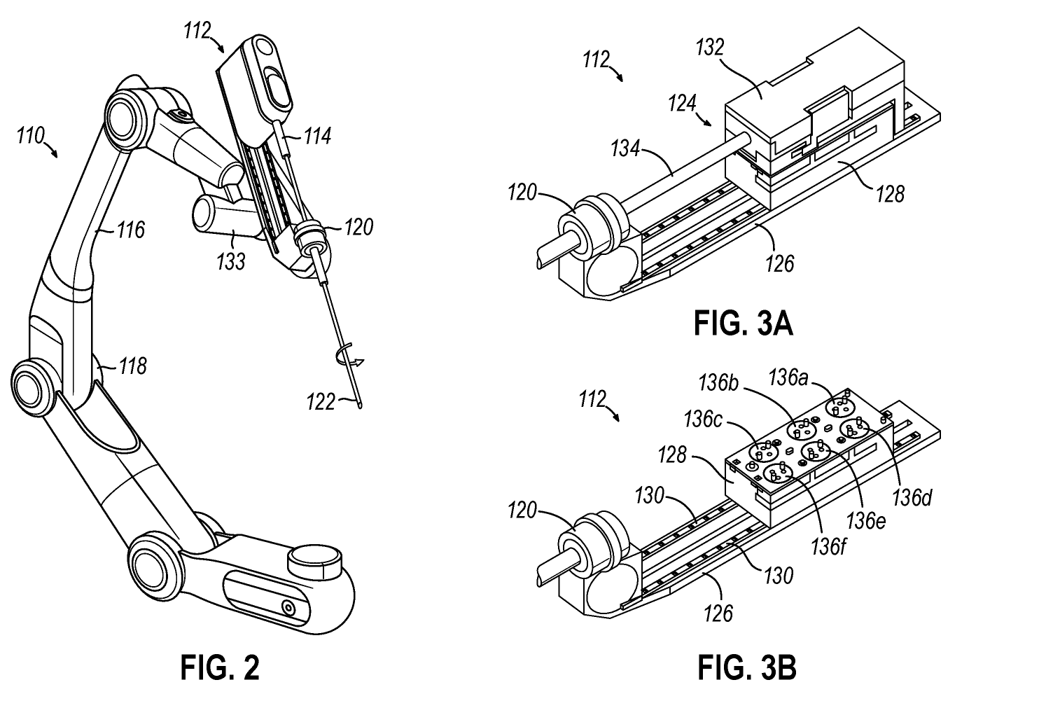

FIG. 2 shows an example of a robotic arm (110), a tool driver (112), and a surgical instrument (114), which may be incorporated into robotic surgical system (10) in place of a robotic arm (16), a tool driver (22), and a surgical instrument (24) that are shown in FIG. 1. Additional examples of robotic arms, tool drivers, and surgical instruments are shown and described in U.S. Pat. No. 10,166,082, entitled “System and Method for Controlling a Robotic Wrist,” issued Jan. 1, 2019, the disclosure of which is incorporated by reference herein, in its entirety.

As shown in FIG. 2, robotic arm (110) includes a plurality of links (116) and a plurality of joints (118) for actuating links (116) relative to one another. Tool driver (112) is attached to the distal end of robotic arm (110). Tool driver (112) includes a cannula (120) coupled to the end of tool driver (112), to receive and guide surgical instrument (114). Surgical instrument (114) may include an endoscope, a laparoscope, a stapler, graspers, an ultrasonic instrument, an RF electrosurgical instrument, or any other suitable kind of instrument. Surgical instrument (114) is inserted into the patient via cannula (120). The distal end of surgical instrument (114) includes an end effector (122). End effector (122) is configured to interact with the patient (e.g., providing visualization, stapling, grasping, ultrasonic cutting and/or sealing, electrosurgical cutting and/or sealing, etc.).

Joints (118) of robotic arm (110) may be actuated to selectively position and orient tool driver (112), which actuates the end effector (122) for robotic surgeries. Joints (118) may include various types, such as a pitch joint or a roll joint, which may substantially constrain the movement of the adjacent links (116) around certain axes relative to other links (116). Each joint (118) represents an independent degree of freedom available to robotic arm (110). A multitude of joints (118) result in a multitude of degrees of freedom, allowing for “redundant” degrees of freedom. Redundant degrees of freedom allow the robotic arms (110) to position their respective end effectors (122) at a specific position, orientation, and trajectory in space using different positions links (116) and angles of joints (118). This allows for the system to position and direct a surgical instrument (114) from a desired point in space while allowing the clinician to move joints (118) into a clinically advantageous position away from the patient to create greater access, while avoiding collisions of robotic arms (110).

FIGS. 3A and 3B show tool driver (112) with and without a tool driver adapter (124), which may also be referred to as a tool base. As shown in FIGS. 3A and 3B, tool driver (112) may include a stage (126) and a carriage (128). Stage (126) includes longitudinal tracks (130). Carriage (128) is slidingly engaged with longitudinal tracks (130). Stage (126) of tool driver (112) may be configured to couple to a distal end (133) of robotic arm (110) such that articulation of robotic arm (110) positions and/or orients tool driver (112) in space. Surgical instrument (114) includes a tool driver adapter (124) at a proximal end and, as noted above, end effector (122) at a distal end. Tool driver adapter (124) includes a handle (132) and a shaft assembly (134) that extends distally from handle (132).

Carriage (128) is configured to couple with tool driver adapter (124). Carriage (128) may drive a set of articulated movements of end effector (122) and/or otherwise actuate end effector (122), such as through a cable system or wires manipulated and controlled by actuated drives. Carriage (128) may include different configurations of actuated drives, including but not limited to motorized rotary axis drives. The plurality of rotary axis drives may be arranged in any suitable manner. As shown in FIG. 3B, carriage (128) of the present example includes six rotary drives (136a-f) arranged in two rows, extending longitudinally along the base of carriage (128). Rotary drives (136a-c) are arranged in a first row that is longitudinally offset from a second row in which rotary drives (136d-f) are arranged. This staggered arrangement of rotary drives (136a-f) may reduce the width of carriage (128) and thereby provide a more compact form factor for tool driver (112). However, rotary drives (136a-f) may be provided in any other suitable arrangement. Moreover, any other suitable kind(s) of drive outputs may be provided by carriage (128), in addition to or in lieu of rotary drives (136a-f).

### II. Examples of Surgical Instruments

### A. Overview

Robotic surgical system (10) includes a limited number of robotic arms (16, 110) onto which scopes and other surgical instruments may be coupled. Given the space constraints of robotic laparoscopic instrumentation, there are competing demands for use of these limited number of robotic arms (16, 110). For example, a surgeon may not want to dedicate a robotic arm to a surgical instrument that provides only suction, only irrigation, or only electrosurgical energy. As a result, it is beneficial for a surgical instrument coupled with one of robotic arms (16, 110) to perform multiple functions during the course of a surgical procedure. Additionally, having a surgical instrument that performs multiple functions reduces or eliminates surgical time associated with exchanging a first surgical instrument with a second surgical instrument providing a different function or capability.

### B. Example of a Surgical Instrument

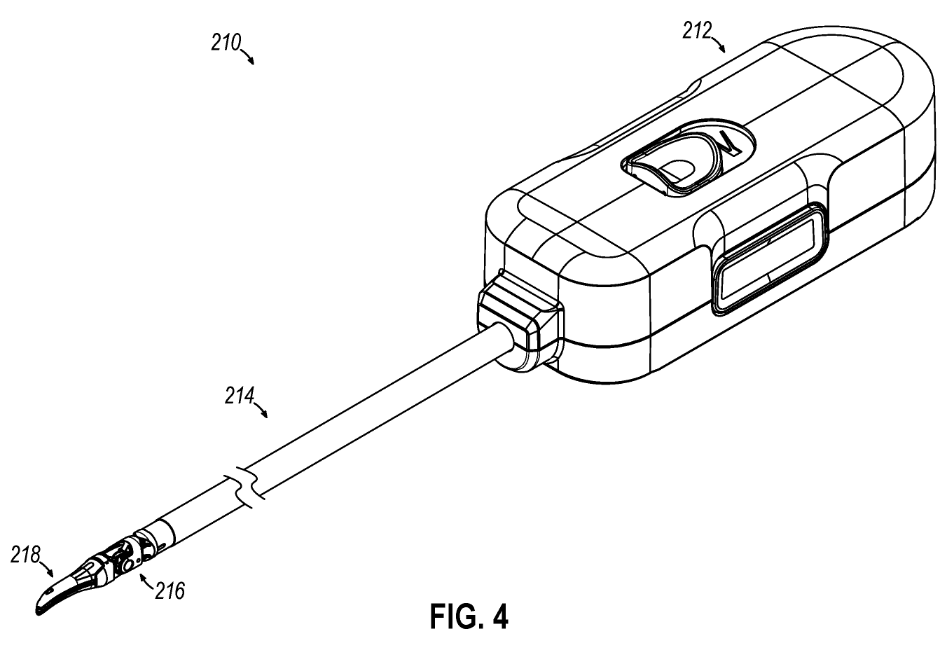

FIG. 4 shows a second example of a surgical instrument (210) configured for use with robotic surgical system (10) including robotic arms (16, 110) and tool drivers (22, 112) of FIGS. 1-2. Surgical instrument (210) may be used in place of surgical instrument (24, 114). As previously described, tool driver (112) includes stage (126) and carriage (128), with carriage (128) being configured to move relative to stage (126) to move surgical instrument (210) relative to patient (P). Surgical instrument (210) includes a body (shown as a tool drive adapter (212)), a shaft assembly (214), an articulation section (216) of shaft assembly (214), and an end effector (218).

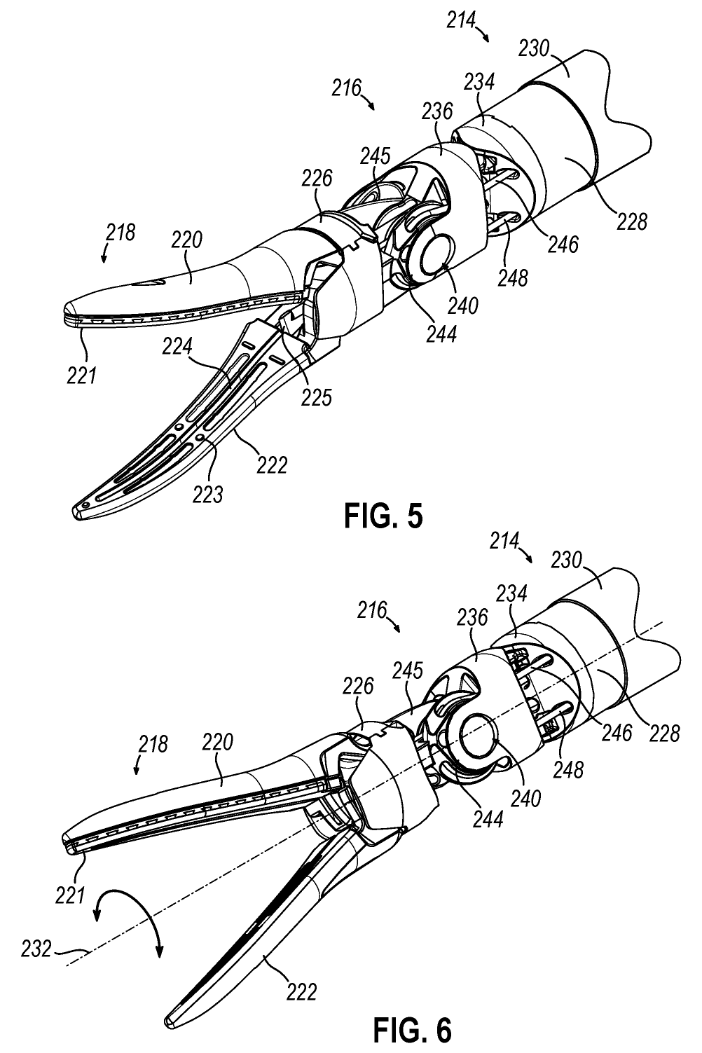

Turning to FIGS. 5-6, end effector (218) includes an upper jaw (220) having an upper electrode surface (221), a lower jaw (222) having a lower electrode surface (223), and a knife member (225) slidably disposed within a knife channel (224) cooperatively defined by upper jaw (220) and lower jaw (222). As will be described in greater detail below, end effector (218) of the current example is configured to grasp tissue with jaws (220, 222), seal tissue by applying bipolar RF energy to tissue via electrodes (221, 223), and sever tissue via knife member (225). In the current example, end effector (218) is suitably coupled to drive inputs (260, 262, 264, 266, 268, 270) (see FIG. 11) via cables and pulleys in order to suitably actuate components of end effector (218) in accordance with the description herein.

Upper jaw (220) and lower jaw (222) are pivotally coupled to each other such that jaws (220, 222) may actuate between an open configuration and a closed configuration in order to grasp tissue. In the current example, jaws (220, 222) are operatively attached to a clevis assembly (226) configured to translate to thereby pivot jaws (220, 222) between the open configuration and the closed configuration. While clevis assembly (226) is utilized in the current example, any other suitable structures may be utilized in order to drive jaws (220, 222) between the open and closed positions as would be apparent to one skilled in the art in view of the teachings herein

Articulation section (216) extends between end effector (218) and an inner shaft (228) of shaft assembly (214). Inner shaft (228) is received within an outer shaft (230) of shaft assembly (214) and configured to selectively rotate about a longitudinal axis (232) within outer shaft (230). In turn, articulation section (216) and end effector (218) are rotatably fixed relative to inner shaft (228) such that articulation section (216) and end effector (218) similarly selectively rotate with inner shaft (228). Such rotation about the longitudinal axis (232) may also be referred to herein as “roll” or “rolling” to position articulation section (216) and end effector (218) as desired for improved angles to manipulate end effector (218) as discussed below in greater detail.

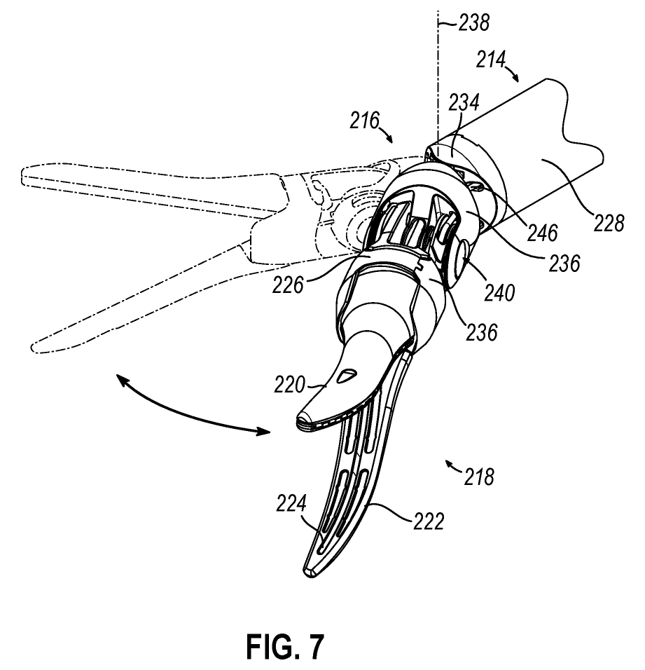

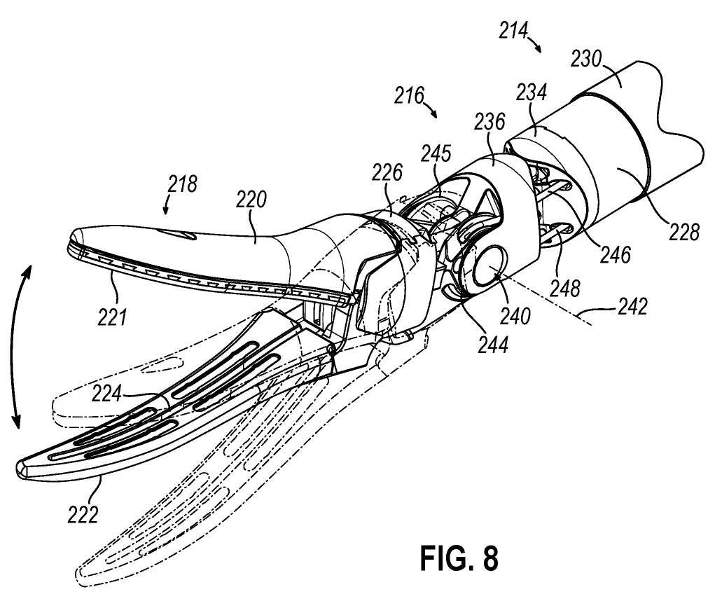

To this end, articulation section (216) is configured to articulate pitch relative to inner shaft (228) and further articulate yaw relative to inner shaft (228) to deflect end effector (218) respectively through a pitch plane as shown in FIG. 7 and a yaw plane as shown in FIG. 8. In the present example, articulation section (216) includes a proximal camming body (234) associated with a distal end of inner shaft (228) and a distal camming body (236) proximally projecting relative to end effector (218). Camming bodies (234, 236) are configured to engage each other as jaws (220, 222) deflect about a pitch axis (238), as shown in FIG. 7.

Articulation section (216) further includes a pivot coupling (240) such that jaws (220, 222) are pivotally coupled to a distal end portion of distal camming body (236). In this respect, pivot coupling (240) is configured to pivot jaws (220, 222) about a yaw axis (242). Pivot coupling (240) more particularly includes a pair of pulleys (244, 245) and a plurality of drive cables (246, 248, 250, 252) (see FIG. 11), which are configured to be collectively driven to pivot jaws (220, 222) together through yaw plane for repositioning jaws (220, 222) as desired. Pulleys (244, 245) and cables (246, 248, 250, 252) are further configured to be collectively driven to pivot jaws (220, 222) relative to each other between the open and closed configurations.

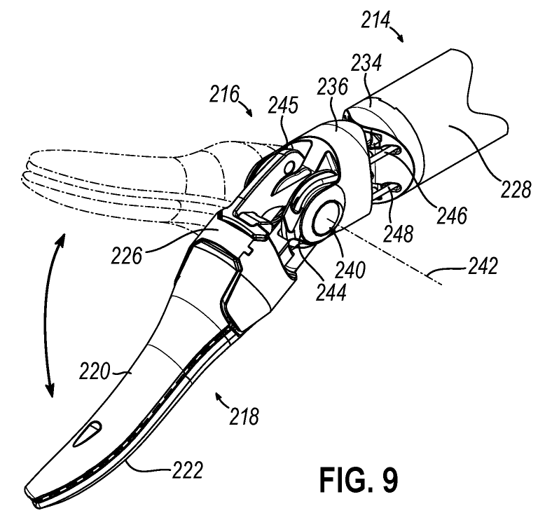

With respect to FIG. 9, while jaws (220, 222) are in the closed configuration, knife member (225) may be driven distally along a path defined by knife channel (224) from a proximal position to a distal position in order to sever tissue grasped by jaws (220, 222). Once knife member (225) reaches the distal position within knife channel (224) in order to suitably sever tissue, knife member (225) may then be retracted within knife channel (224) back into the proximal position. Notably, in the present example, knife member (225) distally extends from an elongate member (253), such as a nitinol tube, which is configured to translate through articulation section (216) for moving knife member (225) as discussed below in greater detail.

Electrode surfaces (221, 223) may be activated during any suitable time at which jaws (220, 222) interact with tissue in order to apply bipolar RF energy to tissue. For example, electrode surfaces (221, 223) may be activated after knife member (225) severs tissue in order to seal the recently severed tissue grasped between jaws (220, 222). As another illustrative example, electrode surfaces (221, 223) may be activated prior to knife member (225) severing tissue. As yet another illustrative example, electrode surface (221, 223) may be activated in order to cauterize tissue without cutting tissue.

In the current example, electrode surface (221) is an electrode body attached on an underside of jaw (220); while jaw (222) is formed from a suitable material in order to act as electrode surface (223). For example, jaw (222) may be formed of a metal material and be in connection with a ground wire; while electrode body forming electrode surface (221) is attached the underside of jaw (220) and in communication with a hot wire. Once suitably activated, RF energy may be transmitted between electrode surfaces (221, 223) in order to further transmit such RF energy through tissue.

Electrode surfaces (221, 223) may have any suitable configuration as would be apparent to one skilled in the art in view of the teachings herein. While in the current example, electrode surfaces (221, 223) are configured to deliver bipolar RF energy to tissue, it should be understood that end effector (218) may be configured to deliver any other suitable type of therapeutic energy to tissue as would be apparent to one skilled in the art in view of the teachings herein.

Electrode surfaces (221, 223) are positioned between remaining portions of upper jaw (220) and lower jaw (222). In certain instances it may be beneficial to include an electrode extending from jaws (220, 222) towards a finer point. It may also be beneficial for end effector (218) to be capable of wedging between closely held tissues and to include a cutting feature.

### III. Various Examples of Tool Accessories for an Instrument End Effector

Described below are various examples of tool accessories (300, 400, 500, 600, 700, 800, 900, 1000, 1100, 1200, 1300) which are attachable to an end effector, such as those discussed above and which may also be referred to below as an instrument end effector (218). Attachment of tool accessory (300, 400, 500, 600, 700, 800, 900, 1000, 1100, 1200, 1300) to instrument end effector (218) may be done by clamping tool accessory (300, 400, 500, 600, 700, 800, 900, 1000, 1100, 1200, 1300) with jaws (220, 222) of instrument end effector (218) and by attaching tool accessory (300, 400, 500, 600, 700, 800, 900, 1000, 1100, 1200, 1300) to any one of jaws (220, 222). A tool end effector (304, 404, 504, 604, 704, 804, 904, 1004, 1104, 1204, 1304) of tool accessory (300, 400, 500, 600, 700, 800, 900, 1000, 1100, 1200, 1300) may then be used to separate and ablate tissue using the previously described articulation and ablation features of instrument end effector (218). A user may selectively attach tool accessory (300, 400, 500, 600, 700, 800, 900, 1000, 1100, 1200, 1300) and may optionally use monopolar or bipolar ablation with tool accessory (300, 400, 500, 600, 700, 800, 900, 1000, 1100, 1200, 1300). While ablation with tool accessory (300, 400, 500, 600, 700, 800, 900, 1000, 1100, 1200, 1300) is described in detail below, this is optional and a user may instead use tool accessory (300, 400, 500, 600, 700, 800, 900, 1000, 1100, 1200, 1300) as an extension of instrument end effector (218).

### A. Example of a First Accessory Tool for an Instrument End Effector

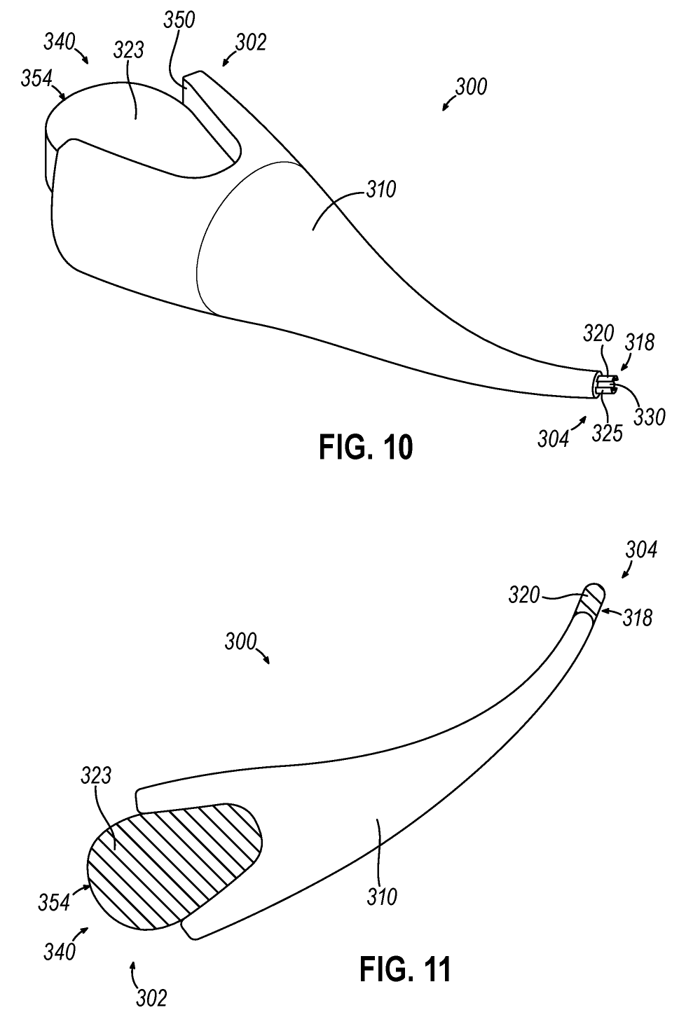

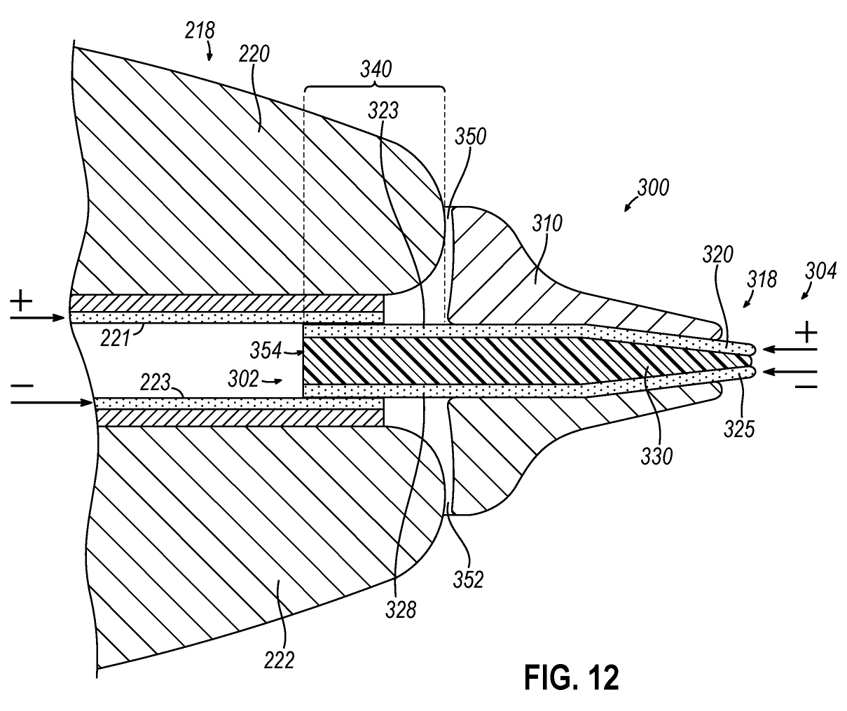

FIGS. 10-12 depict an example of a first tool accessory (300) for attaching to a distal end of instrument end effector (218). First tool accessory (300) includes a proximally positioned coupling adapter (302), a distally positioned tool end effector (304), and a shroud (310) extending therebetween. Tool end effector (304) of the present example more particularly includes a distal end (318), an upper tool electrode (320), and a lower tool electrode (325). An insulator core (330) extends through each of coupling adapter (302), shroud (310), and tool end effector (304). Coupling adapter (302) of the present example more particularly includes a grip portion (340) as discussed below in greater detail. Shroud (310) acts as an electrical insulator and surrounds middle portions of upper and lower tool electrodes (320, 325). Shroud (310) is shaped to cover and extend from a distal end of upper jaw (220) and lower jaw (222) such that there is a continuous covering of upper and lower electrodes (320, 325) by jaws (220, 222) and shroud (310), as shown. Shroud (310) may be a rigid material such that it is able to maintain a shape or may be a pliable material such that it is able to bend and maintain a bent shape.

As shown, shroud (310) surrounds upper and lower tool electrodes (320, 325). A distal portion of insulator core (330) is sandwiched between upper tool electrode (320) and lower tool electrode (325) such that distal portion of insulator core (330) electrically isolates upper and lower electrodes (320, 325) but for distal portions of upper and lower electrodes (320, 325), as shown. Upper and lower tool electrodes (320, 325) extend distally beyond shroud (310) and beyond distal portion of insulator core (330) to thereby be exposed to the surrounding environment. A distal-most end of distal portion of insulator core (330) is therefore proximal distal-most ends of upper and lower tool electrodes (320, 325) such that upper and lower electrodes (320, 325) are in a direct line-of-sight with each other and thus are able to collectively achieve bipolar ablation at their distal-most ends. Upper and lower electrodes (320, 325) may be tapered or angled towards each other in a distal direction such that they are furthest apart at a proximal portion and closest at a distal portion, mainly at the distal-most ends. As shown, upper and lower electrodes (320, 325) may each include proximal portions that run parallel to one another before tapering to allow for parallel portions of electrode surfaces (221, 223) to evenly grip along upper grip surface (323) and lower grip surface (328). Insulator core (330) may be shaped accordingly to allow for such a separation between upper and lower electrodes (320, 325), notably, thicker along a proximal portion than along a distal portion.

As shown, upper and lower tool electrodes (320, 325) extend proximally beyond shroud (310) to thereby be engaged, such as by being clamped, by upper and lower jaws (220, 222) of instrument end effector (218). Specifically, upper jaw (220) is received within an upper pocket (350) of coupling adapter (302), whereas lower jaw (222) is received within a lower pocket (352) of coupling adapter (302). A mount tab (354) proximally extends from shroud (310) between upper and lower pockets (350, 352) includes grip portion (340) thereon, which is shown as being the portion proximal to adjacent shroud (310) and thus available to be gripped via upper and lower jaws (220, 222). Tool accessory (300) is able to electrically communicate RF energy from electrode surfaces (221, 223) to respective upper and lower tool electrodes (320, 325) through direct contact with upper grip surface (323) and lower grip surface (328), respectively. Grip surfaces (323, 328) are shown in the present example on mount tab (354) and may have a rougher surface finish than electrode surfaces (221, 223) to promote grip between the two. As shown grip portion (340) is incorporated into upper and lower tool electrodes (320, 325) to thus achieve two separate objectives; holding tool accessory (300) relative to end effector (218) and the aforementioned electrical communication between electrode surfaces (221, 223) and upper and lower tool electrodes (320, 325). However, as described below, this dual function may be separated such that individual components accomplish separate functions.

Insulator core (330) therefore as a proximal portion positioned between electrically conductive grip surfaces (323, 328) at upper and lower tool electrodes (320, 325) for electrical isolation between upper and lower tool electrodes (320, 325) at coupling adapter (302). Additionally, insulator core (330) may also include a separate proximal portion (not shown) which extends proximally beyond upper and lower tool electrodes (320, 325). This proximal portion may be used to hold tool accessory (300) to end effector (218) such that upper and lower tool electrodes (320, 325) simply conduct electricity from electrode surfaces (221, 223) rather than aid in holding.

As detailed in FIGS. 10-11, shroud (310) and upper and lower tool electrodes (320, 325) define an arcuate path while extending distally. While this arcuate path is shown being directed laterally, it can instead be directed upwards (into the page) or in a combination of lateral and upwards. In some examples, tool accessory (300) can be flipped 180 degrees relative to end effector (218) such that rather than arching to the left and/or upwards, tool accessory (300) can arch to the right and/or downwards. The orientation may be selectable by a user based on present needs. Further, shroud (310) and upper and lower tool electrodes (320, 325) may be pliable/malleable such that shroud (310) and upper and lower tool electrodes (320, 325) are able to be bent by a user and to maintain the bent shape.

Distal end (318) of tool accessory (300) tapers down to form a wedge shape. Such a shape may be used to wedge distal end (318) into tissues to thereby separate the tissues. Further, distal end (318) may include a cutting edge capable of cutting adjacent tissues. As shown, upper and lower tool electrodes (320, 325) are capable of bipolar ablation. Additionally, either of upper or lower tool electrodes (320, 325) may be capable of monopolar ablation by isolating the other upper or lower tool electrode (320, 325). From this, a user may use distal end (318) to wedge into tissue, cut the tissue, and perform bi/monopolar ablation on the tissue in parallel or series steps.

### B. Example of a Second Tool Accessory for an Instrument End Effector

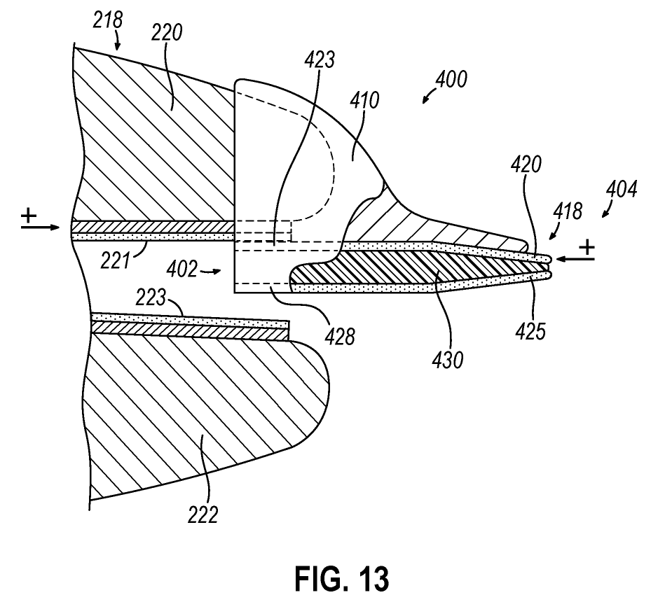

FIG. 13 depicts an example of a second tool accessory (400) which is substantially similar to first tool accessory (400) with a coupling adapter (402), a tool end effector (404), and a shroud (410) in form and function but for the below differences. As shown, second tool accessory (400) can attached to a single jaw (220, 222) of end effector (218) rather than needing to be sandwiched between jaws (220, 222). Shroud (410) includes a proximally-facing opening sized and shaped to fit upper jaw (220). Shroud (410) is able to selectively affix to upper jaw (220) and, through this fixation, contact upper tool electrode (420) to upper electrode surface (221). As shown, end effector (218) in the open configuration may still transmit monopolar RF energy to tool accessory (400). This may be useful when end effector (218) is used to spread tissue while also wanting to perform monopolar ablation. Bipolar ablation may also be performed once lower electrode surface (223) contacts lower tool electrode (325) which may further enhance the fixation between second tool accessory (400) and instrument end effector (218).

### C. Example of a Third Tool Accessory for an Instrument End Effector

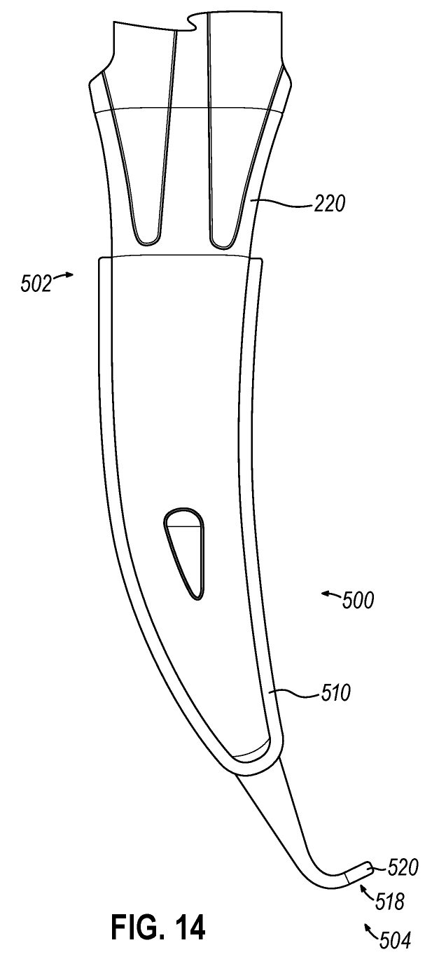

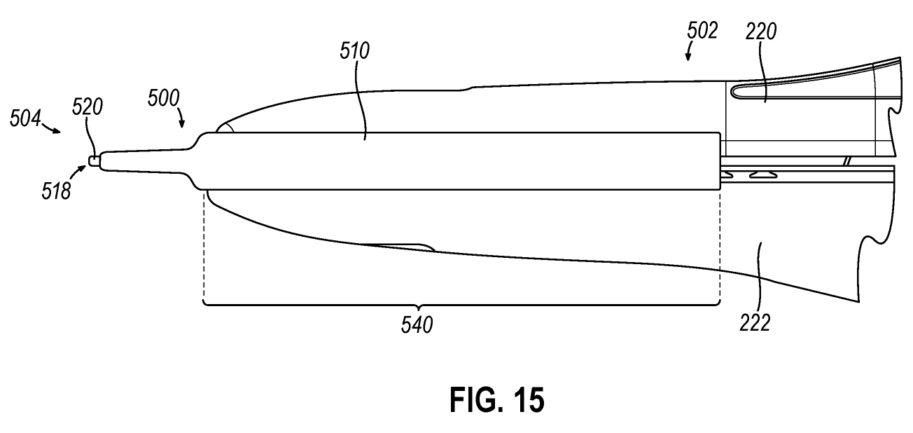

FIGS. 14-15 depict a example of a third tool accessory (500) extending substantially within end effector (218). Third tool accessory (500) is substantially similar to tool accessories (300, 400) with a coupling adapter (502), a tool end effector (504), and a shroud (510) in form and function but for the below differences. Third tool accessory (500) includes shroud (510) which wraps around a perimeter of end effector (218) to thereby shield exposed portions of electrode surfaces (221, 223) to thereby inhibit lateral electrical leakage. Shroud (510) may be shaped to be pressed onto upper jaw (220) to thereby be affixed to a single jaw. Shroud (510) includes a curved or hooked distal end (518) having an exposed tool electrode (520).

### D. Example of a Fourth Tool Accessory for an End Effector

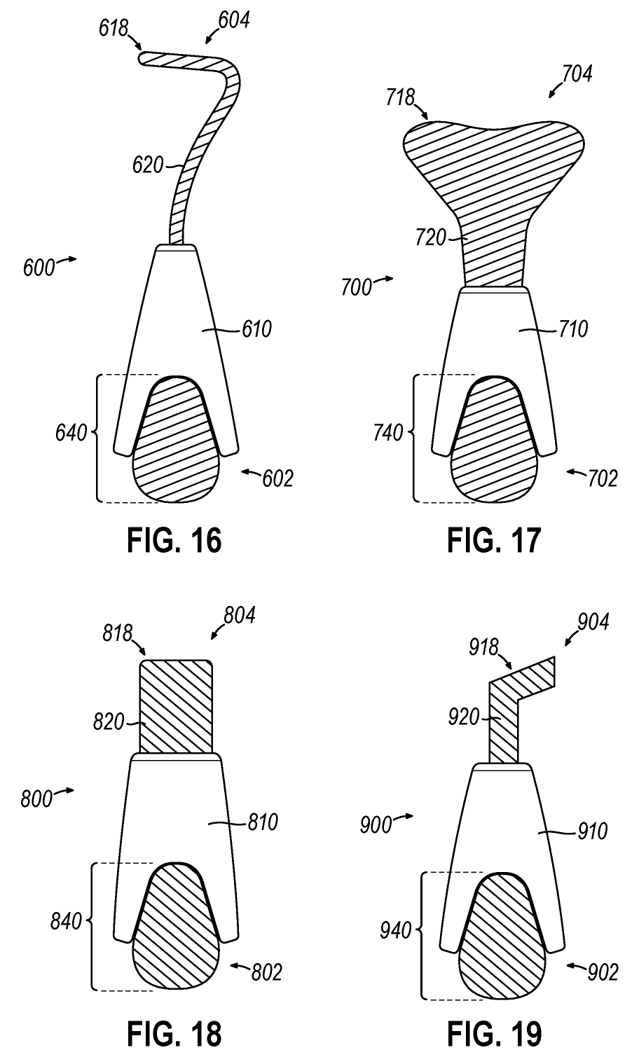

FIG. 16 depicts an example of a fourth tool accessory (600) which is substantially similar to tool accessories (300, 400, 500) with a coupling adapter (602), a tool end effector (604), and a shroud (610) in form and function but for the below differences. Fourth tool accessory (600) may include proximal grip portion (640) surrounded by a shroud (610) with a distal end (618) extending therefrom. Distal end (618) includes upper tip electrode (620) which is shaped as an elongated hook having a rounded end. Distal end (618) may be used for hooking onto target tissue.

### E. Example of a Fifth Tool Accessory for an Instrument End Effector

FIG. 17 depicts an example of a fifth tool accessory (700) which is substantially similar to tool accessories (300, 400, 500, 600) with a coupling adapter (702), a tool end effector (704), and a shroud (710) in form and function but for the below differences. Fifth tool accessory (700) may include proximal grip portion (740) surrounded by a shroud (710) with a distal end (718) extending therefrom. Distal end (718) includes upper tip electrode (720) which is shaped as a fin and extends laterally from shroud (710). Distal end (618) may be used for pressing against large portions of target tissue.

### F. Example of a Sixth Tool Accessory for an Instrument End Effector

FIG. 18 depicts an example of a sixth tool accessory (800) which is substantially similar to tool accessories (300, 400, 500, 600, 700) with a coupling adapter (802), a tool end effector (804), and a shroud (810) in form and function but for the below differences. Sixth tool accessory (800) may include proximal grip portion (840) surrounded by a shroud (810) with a distal end (818) extending therefrom. Distal end (818) includes upper tip electrode (820) which is shaped as a rectangle and extends distally from shroud (810). Distal end (818) may be used for pressing against large portions of target tissue.

### G. Example of a Seventh Tool Accessory for an Instrument End Effector

FIG. 19 depicts an example of a seventh tool accessory (900) which is substantially similar to tool accessories (300, 400, 500, 600, 700, 800) with a coupling adapter (902), a tool end effector (904), and a shroud (910) in form and function but for the below differences. Seventh tool accessory (900) may include proximal grip portion (940) surrounded by a shroud (910) with a distal end (918) extending therefrom. Distal end (918) includes upper tip electrode (920) which is angled and extends distally from shroud (910). Distal end (918) may be used for accessing portions of tissue otherwise blind to end effector (218).

### H. Example of an Eighth Tool Accessory for an Instrument End Effector

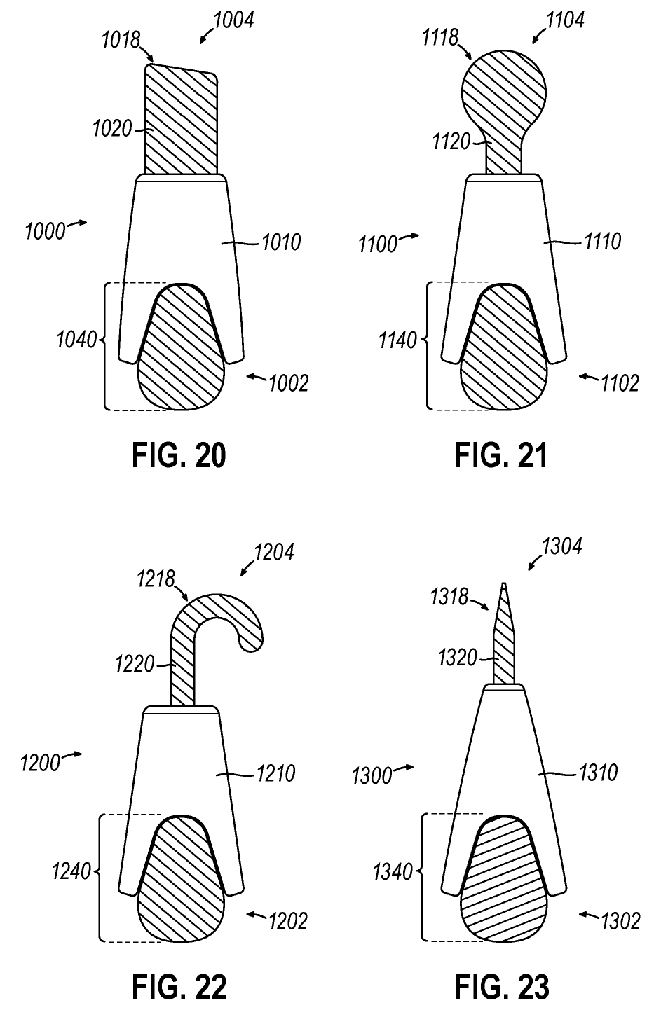

FIG. 20 depicts an example of an eighth tool accessory (1000) which is substantially similar to tool accessories (300, 400, 500, 600, 700, 800, 900) with a coupling adapter (1002), a tool end effector (1004), and a shroud (1010) in form and function but for the below differences. Eighth tool accessory (1000) may include proximal grip portion (1040) surrounded by a shroud (1010) with a distal end (1018) extending therefrom. Distal end (1018) includes upper tip electrode (1020) which is shaped as a right trapezoid and extends distally from shroud (1010). Distal end (1018) may be used for pressing against large portions of target tissue and includes a sharp angle and a dull angle.

### I. Example of a Ninth Tool Accessory for an Instrument End Effector

FIG. 21 depicts an example of a ninth tool accessory (1100) which is substantially similar to tool accessories (300, 400, 500, 600, 700, 800, 900, 1000) with a coupling adapter (1102), a tool end effector (1104), and a shroud (1110) in form and function but for the below differences. Ninth tool accessory (1100) may include proximal grip portion (1140) surrounded by a shroud (1110) with a distal end (1118) extending therefrom. Distal end (1118) includes upper tip electrode (1120) which is shaped to include a distal circle and extends distally from shroud (1110). Distal end (1118) may be used for pressing against large portions of target tissue.

### J. Example of a Tenth Tool Accessory for an Instrument End Effector

FIG. 22 depicts an example of a tenth tool accessory (1200) which is substantially similar to tool accessories (300, 400, 500, 600, 700, 800, 900, 1000, 1100) with a coupling adapter (1202), a tool end effector (1204), and a shroud (1210) in form and function but for the below differences. Tenth tool accessory (1200) may include proximal grip portion (1240) surrounded by a shroud (1210) with a distal end (1218) extending therefrom. Distal end (1218) includes upper tip electrode (1220) which is shaped to include a “J” hook and extends distally from shroud (1210). Distal end (1218) may be used for hooking portions of target tissue and accessing blind tissue.

### K. Example of an Eleventh Tool Accessory for an Instrument End Effector

FIG. 23 depicts an example of an eleventh tool accessory (1300) which is substantially similar to tool accessories (300, 400, 500, 600, 700, 800, 900, 1000, 1100, 1200) with a coupling adapter (1302), a tool end effector (1304), and a shroud (1310) in form and function but for the below differences. Eleventh tool accessory (1300) may include proximal grip portion (1340) surrounded by a shroud (1310) with a distal end (1318) extending therefrom. Distal end (1318) includes upper tip electrode (1320) which is shaped as a pointed tip and extends distally from shroud (1310). Distal end (1318) may be used for piercing portions of target tissue and accessing blind tissue.

### IV. Method of Affixing Tool Accessory to an Instrument End Effector

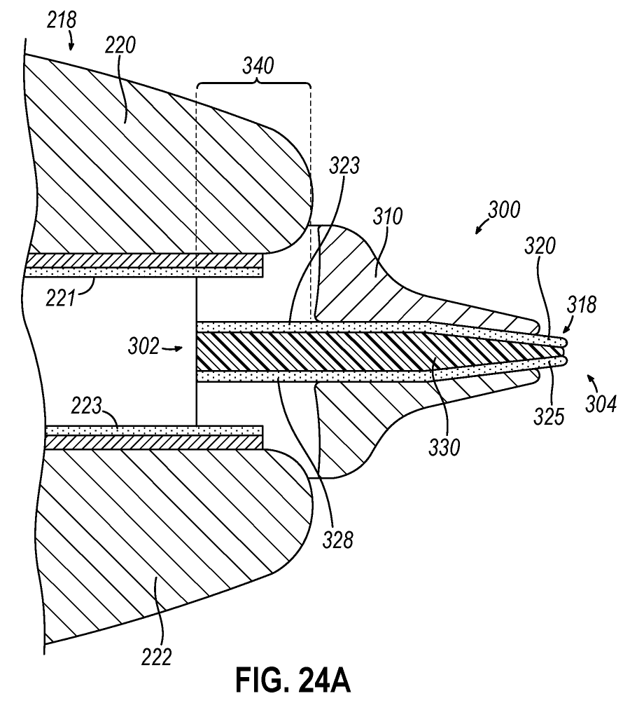

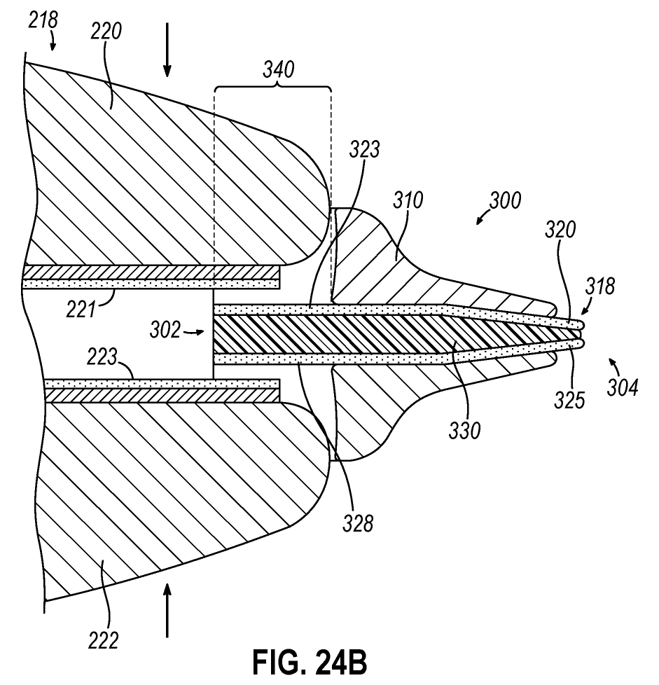

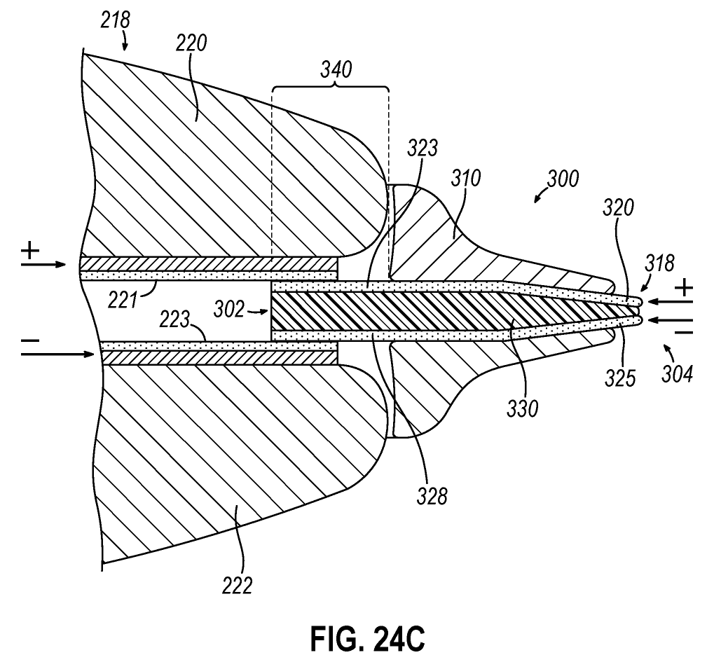

FIGS. 24A-24C depict a method of affixing any one of tool accessories (300, 400, 500, 600, 700, 800, 900, 1000, 1100, 1200, 1300) to instrument end effector (218). As shown in FIG. 24A, tool accessory (300) is positioned within end effector (218) while in the open configuration such that tool accessory (300) is between jaws (220, 222). Accordingly, end effector (218) in the open configuration defines a gap between opposing jaws (220, 222) for which grip portion (340) is sized to fit within. Alignment and depth between end effector (218) and tool accessory (300) may be accomplished or aided using shroud (310). FIG. 24B depicts jaws (220, 222) coming together to thus reduce the gap and help to vertically centralize tool accessory (300). FIG. 24C shows each jaw (220, 222) in contact with tool accessory (300) such that upper electrode surface (221) electrically contacts upper tool electrode (320) and lower electrode surface (223) contacts lower tool electrode (325). As shown, a voltage potential may then be applied to tool accessory (300).

### V. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) an accessory tool, comprising: (i) a shroud, wherein at least a portion of the shroud is formed of an electrical insulator, (ii) a coupling adapter proximally extending from the shroud, including: (A) an upper grip portion configured to removably secure with an instrument end effector, and (B) an upper grip surface configured to contact an upper electrode of the instrument end effector, wherein the upper grip surface is electrically conductive, and (iii) a tool end effector distally extending from the shroud, wherein the tool end effector includes an upper tool electrode in electrical communication with the upper grip surface.

### Example 2

The surgical instrument of Example 1, wherein the coupling adapter includes an upper pocket and a lower pocket configured to respectively receive an upper jaw of the instrument end effector and a lower jaw of the instrument end effector.

### Example 3

The surgical instrument of any one or more of Examples 1 through 2, wherein the upper and lower pockets are integrally formed with the shroud.

### Example 4

The surgical instrument of any one or more of Examples 1 through 3, wherein the coupling adapter includes a mount tab proximally extending from the shroud and positioned between the upper and lower pockets, wherein the mount tab is configured to be compressed between the upper and lower jaws of the instrument end effector for removable securement therewith, and wherein the upper grip portion and the upper grip surface are positioned on the mount tab.

### Example 5

The surgical instrument of any one or more of Examples 1 through 4, wherein the coupling adapter further includes: (A) a lower grip portion configured to removably secure with the instrument end effector, and (B) a lower grip surface configured to contact a lower electrode of the instrument end effector, wherein the lower grip surface is electrically conductive.

### Example 6

The surgical instrument of Example 5, wherein the coupling adapter further includes a proximal insulator portion positioned between the upper grip portion and the lower grip portion and configured to electrically insulate the upper grip surface from the lower grip surface.

### Example 7

The surgical instrument of any one or more of Examples 5 through 6, wherein the tool end effector further includes a lower tool electrode distally extending from the shroud, wherein the lower tool electrode is in electrical communication with the lower grip surface.

### Example 8

The surgical instrument of Example 7, wherein the tool end effector further includes a distal insulator portion positioned between the upper tool electrode and the lower tool electrode and configured to electrically insulate the upper grip surface from the lower grip surface, and wherein the distal insulator portion distally extends beyond the shroud.

### Example 9

The surgical instrument of Example 8, wherein the coupling adapter further includes a proximal insulator portion positioned between the upper grip portion and the lower grip portion and configured to electrically insulate the upper grip surface from the lower grip surface, wherein the upper grip portion, the lower grip portion, and the proximal insulator portion proximally extend from the shroud and are thus configured to be sandwiched between the upper and lower jaws of the end effector.

### Example 10

The surgical instrument of Example 9, wherein the proximal insulator portion has a proximal insulator thickness in a direction perpendicular to the upper grip surface, wherein the distal insulator portion has a distal insulator thickness in the direction perpendicular to the upper grip surface, and wherein the proximal insulator thickness is larger than the distal insulator thickness.

### Example 11

The surgical instrument of Example 10, wherein the accessory tool includes an insulator core formed of an electrical insulator, and wherein the insulator core includes each of the proximal insulator portion and the distal insulator portion.

### Example 12

The surgical instrument of any one or more of Examples 1 through 11, wherein the upper tool electrode and the shroud follow an arcuate path extending from the upper grip portion.

### Example 13

The surgical instrument of any one or more of Examples 1 through 12, wherein the shroud and the upper tool electrode taper distally towards a distal end.

### Example 14

The surgical instrument of any one or more of Examples 1 through 13, wherein the upper tool electrode includes a sharp distal end configured to cut tissue.

### Example 15

The surgical instrument of claim 1, further comprising: (a) a shaft assembly; and (b) an instrument end effector distally extending from the shaft assembly and including: (i) an upper jaw having an upper electrode, and (ii) a lower jaw, wherein the upper and lower jaws are configured to selectively move relative to each other from an open configured toward a closed configuration for removably securing the accessory tool therebetween.

### Example 16

A surgical instrument, comprising: (a) a shaft assembly; (b) an instrument end effector distally extending from the shaft assembly and including: (i) an upper jaw having an upper electrode, and (ii) a lower jaw, wherein the upper and lower jaws are configured to selectively move relative to each other from an open configured toward a closed configuration; and (c) an accessory tool configured to removably secure against the upper and lower jaws, comprising: (i) a shroud, wherein at least a portion of the shroud is formed of an electrical insulator, (ii) a coupling adapter proximally extending from the shroud, including: (A) an upper grip portion configured to removably secure against the upper jaw, (B) an upper grip surface configured to contact the upper electrode, wherein the upper grip surface is electrically conductive, (C) an upper pocket configured to receive the upper jaw, (D) a lower grip portion configured to removably secure against the lower jaw, (E) a lower grip surface configured to contact the lower electrode, wherein the lower grip surface is electrically conductive, and (F) a lower pocket configured to receive the lower jaw, and (iii) a tool end effector distally extending from the shroud, wherein the tool end effector includes: (A) an upper tool electrode in electrical communication with the upper grip surface, and (B) a lower tool electrode in electrical communication with the lower grip surface.

### Example 17

The surgical instrument of Example 16, wherein the coupling adapter further includes a mount tab proximally extending from the shroud and positioned between the upper and lower pockets, wherein the mount tab is configured to be compressed between the upper and lower jaws of the instrument end effector for removable securement therewith.

### Example 18

The surgical instrument of any one or more of Examples 16 through 17, wherein the upper grip portion and the upper grip surface are positioned on the mount tab, and wherein the lower grip portion and the lower grip surface are positioned on the mount tab.

### Example 19

A method of electrically connecting a first electrode of an instrument end effector of a surgical instrument to an accessory tool, the method including: (a) selectively affixing the accessory tool to a first jaw of the instrument end effector to thereby extend the accessory tool distally beyond the instrument end effector; (b) placing the first electrode of the instrument end effector in electrical communication with a tool electrode of the accessory tool; and (c) extending the tool electrode distally beyond the first electrode of the instrument end effector.

### Example 20

The method of Example 19, wherein selectively affixing the accessory tool to the first jaw includes sandwiching the accessory tool between the first jaw and a second jaw of the instrument end effector.

### VI. Miscellaneous

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator. Moreover, terms such as “upper” and “lower” are merely spatial terms relative to the figures and are not intended to unnecessarily limit the invention described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of instruments having RF electrodes, it should be understood that various teachings herein may be readily applied to a variety of other types of devices. By way of example only, the various teachings herein may be readily applied to other types of surgical instruments including tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. A surgical instrument, comprising:
a. an accessory tool, comprising:
i. a shroud, wherein at least a portion of the shroud is formed of an electrical insulator,
ii. a coupling adapter proximally extending from the shroud, including:
A. an upper grip portion configured to removably secure with an instrument end effector, and
B. an upper grip surface configured to contact an upper electrode of the instrument end effector, wherein the upper grip surface is electrically conductive, and
iii. a tool end effector distally extending from the shroud, wherein the tool end effector includes an upper tool electrode in electrical communication with the upper grip surface.

2. The surgical instrument of claim 1, wherein the coupling adapter includes an upper pocket and a lower pocket configured to respectively receive an upper jaw of the instrument end effector and a lower jaw of the instrument end effector.

3. The surgical instrument of claim 2, wherein the upper and lower pockets are integrally formed with the shroud.

4. The surgical instrument of claim 2, wherein the coupling adapter includes a mount tab proximally extending from the shroud and positioned between the upper and lower pockets, wherein the mount tab is configured to be compressed between the upper and lower jaws of the instrument end effector for removable securement therewith, and wherein the upper grip portion and the upper grip surface are positioned on the mount tab.

5. The surgical instrument of claim 1, wherein the coupling adapter further includes:
A. a lower grip portion configured to removably secure with the instrument end effector, and
B. a lower grip surface configured to contact a lower electrode of the instrument end effector, wherein the lower grip surface is electrically conductive.

6. The surgical instrument of claim 5, wherein the coupling adapter further includes a proximal insulator portion positioned between the upper grip portion and the lower grip portion and configured to electrically insulate the upper grip surface from the lower grip surface.

7. The surgical instrument of claim 5, wherein the tool end effector further includes a lower tool electrode distally extending from the shroud, wherein the lower tool electrode is in electrical communication with the lower grip surface.

8. The surgical instrument of claim 7, wherein the tool end effector further includes a distal insulator portion positioned between the upper tool electrode and the lower tool electrode and configured to electrically insulate the upper grip surface from the lower grip surface, and wherein the distal insulator portion distally extends beyond the shroud.

9. The surgical instrument of claim 8, wherein the coupling adapter further includes a proximal insulator portion positioned between the upper grip portion and the lower grip portion and configured to electrically insulate the upper grip surface from the lower grip surface, wherein the upper grip portion, the lower grip portion, and the proximal insulator portion proximally extend from the shroud and are thus configured to be sandwiched between the upper and lower jaws of the end effector.

10. The surgical instrument of claim 9, wherein the proximal insulator portion has a proximal insulator thickness in a direction perpendicular to the upper grip surface, wherein the distal insulator portion has a distal insulator thickness in the direction perpendicular to the upper grip surface, and wherein the proximal insulator thickness is larger than the distal insulator thickness.

11. The surgical instrument of claim 10, wherein the accessory tool includes an insulator core formed of an electrical insulator, and wherein the insulator core includes each of the proximal insulator portion and the distal insulator portion.

12. The surgical instrument of claim 1, wherein the upper tool electrode and the shroud follow an arcuate path extending from the upper grip portion.

13. The surgical instrument of claim 1, wherein the shroud and the upper tool electrode taper distally towards a distal end.

14. The surgical instrument of claim 1, wherein the upper tool electrode includes a sharp distal end configured to cut tissue.

15. The surgical instrument of claim 1, further comprising:
a. a shaft assembly; and
b. an instrument end effector distally extending from the shaft assembly and including:
i. an upper jaw having an upper electrode, and
ii. a lower jaw, wherein the upper and lower jaws are configured to selectively move relative to each other from an open configured toward a closed configuration for removably securing the accessory tool therebetween.

16. A surgical instrument, comprising:
a. a shaft assembly;
b. an instrument end effector distally extending from the shaft assembly and including:
i. an upper jaw having an upper electrode, and
ii. a lower jaw, wherein the upper and lower jaws are configured to selectively move relative to each other from an open configured toward a closed configuration; and
c. an accessory tool configured to removably secure against the upper and lower jaws, comprising:
i. a shroud, wherein at least a portion of the shroud is formed of an electrical insulator,
ii. a coupling adapter proximally extending from the shroud, including:
A. an upper grip portion configured to removably secure against the upper jaw,
B. an upper grip surface configured to contact the upper electrode, wherein the upper grip surface is electrically conductive,
C. an upper pocket configured to receive the upper jaw,
D. a lower grip portion configured to removably secure against the lower jaw,
E. a lower grip surface configured to contact the lower electrode, wherein the lower grip surface is electrically conductive, and
F. a lower pocket configured to receive the lower jaw, and
iii. a tool end effector distally extending from the shroud, wherein the tool end effector includes:
A. an upper tool electrode in electrical communication with the upper grip surface, and
B. a lower tool electrode in electrical communication with the lower grip surface.

17. The surgical instrument of claim 16, wherein the coupling adapter further includes a mount tab proximally extending from the shroud and positioned between the upper and lower pockets, wherein the mount tab is configured to be compressed between the upper and lower jaws of the instrument end effector for removable securement therewith.

18. The surgical instrument of claim 17, wherein the upper grip portion and the upper grip surface are positioned on the mount tab, and wherein the lower grip portion and the lower grip surface are positioned on the mount tab.

19. A method of electrically connecting a first electrode of an instrument end effector of a surgical instrument to an accessory tool, the method including:
a. selectively affixing the accessory tool to a first jaw of the instrument end effector to thereby extend the accessory tool distally beyond the instrument end effector;
b. placing the first electrode of the instrument end effector in electrical communication with a tool electrode of the accessory tool; and
c. extending the tool electrode distally beyond the first electrode of the instrument end effector.

20. The method of claim 19, wherein selectively affixing the accessory tool to the first jaw includes sandwiching the accessory tool between the first jaw and a second jaw of the instrument end effector.
